(19) United States
(12) Patent Application Publication
(10) Pub. No.: US 2006/0047182 A1
(43) Pub. Date: Mar. 2, 2006

(54) Combination male sexual organ suction and artificial vagina stimulator device

(76) Inventor: Jack Jenkins Cochran, Tarboro, NC (US)

Correspondence Address: Jack Jenkins Cochran, 412 Holly Court, Tarboro, NC, 27886, US

(21) Appl. No.: 10/932,645

(22) Filed: Sep. 2, 2004

(51) Int. Cl. A61F 5/00

(52) U.S. Cl. 600/38

(57) ABSTRACT

A device for erotically stimulating the male sexual organ and for obtaining an erection by males with Erectile Dysfunction Disorder includes a soft plastic cylindrical container having an open end which forms a cavity for receiving the male sexual organ. The device further includes unattached free-moving membrane-like material within which tap water is sealed inside and said material thus inserted, entirely within the said container. The device is the only device in the medical and sexual aid fields that can easily be assembled at home and provides simultaneous sucking action and realistic-like membranous tactile sensation to the male sexual organ replicating the female vagina.

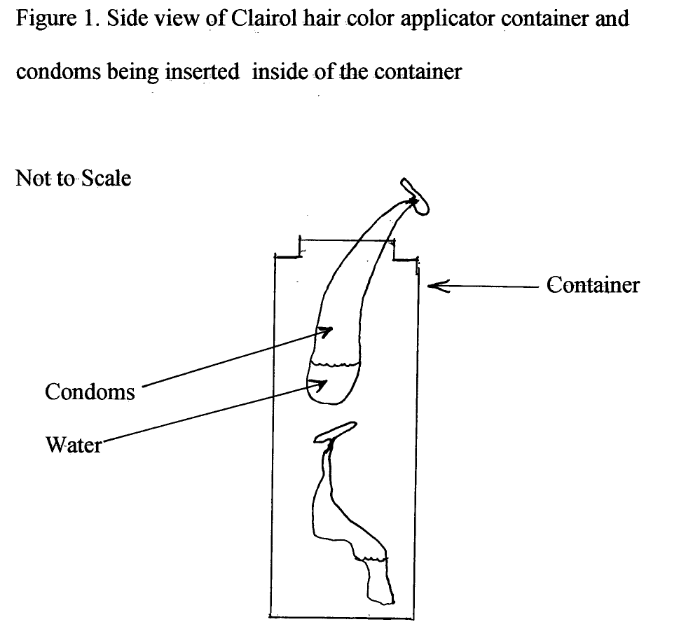

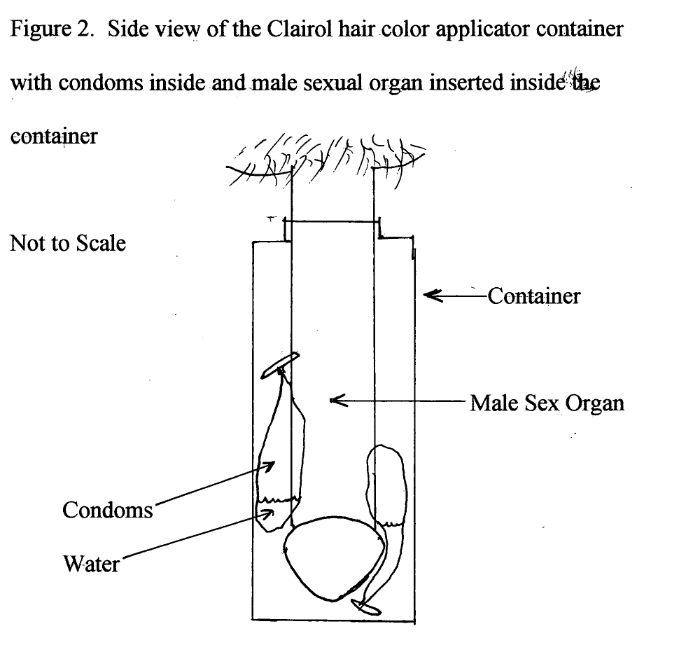

### CROSS REFERENCE TO RELATED APPLICATIONS

NA

### STATEMENT OF FEDERALLY SPONSORED RESEARCH

NA

### REFERENCE TO “SEQUENCE LISTING”

NA

### DETAILED DESCRIPTION

The present invention is used to erotically stimulate the male sexual organ and as an aid for males with EDD to obtain an erection to accomplish sexual intercourse. One takes an approximate six-inch long, 2-inch diameter, soft plastic Clairol hair color, applicator container or equivalent and removes and discards the screw-on top. The said container can be obtained from most hair care or beauty supply stores. Then one takes two condoms, unrolls each condom, inserts about one average mouthful of tap water into each said condom and ties off the end of each said condom, sealing the tap water within each said condom. One then takes each said condom with water sealed inside and inserts them consecutively into the open end of the said container and thence entirely within the said container. One then inserts some lubricant such as hand/body lotion or KY Jelly into the said container. One then inserts one's flaccid or semi-flaccid male sexual organ and begins to squeeze the container creating a sucking action on the male sexual organ. As the male sexual organ begins to become erect one begins to pump the container up and down as males typically do while masturbating. One simultaneously squeezes the said container while pumping the said container up and down to provide the best erotic stimulation. The male organ moves inside the said container and is erotically stimulated by the membrane-like free-floating condoms, which replicates penetration inside the female vagina. The free-floating condoms provide a variable ever-changing tactile surface for the male organ. The said container provides a comfortable and pleasurable tight fit around the base of the shaft of the male sexual organ. One continues this motion until orgasm is reached. Before the male sexual organ is inserted inside the said container, one may optionally heat the said container with the said water-filled condoms inside the said container for about 10 to 15 seconds in a microwave oven to obtain an even better simulation of the female vagina. As with all male organ suction devices one should not maintain continuous suction on the male sexual organ for more than about ten minutes or when the device becomes uncomfortable to prevent injury. For males with larger male organ shafts, one can optionally enlarge the opening or mouth of the said container for a more comfortable fit by heating an heating tool such as the wooden-handle metal-head heating tool found in most vinyl repair kits or an equivalent tool. The present device is easily cleaned by taking the condoms out of the container and rinsing both the condoms and container with hot tap water. The water sealed inside the said condoms is kept inside of the said condoms indefinitely and is not removed during cleaning.

### BACKGROUND OF THE INVENTION

In order for males to gain satisfactory sexual release there are only two methods, actual sexual intercourse with a partner with all the accompanying risks of sexually transmitted diseases and pregnancy, and masturbation of the male organ by oneself.

There are two types of antecedents to the present invention, hard-plastic male organ suction devices from the medical and sex novelty fields which are commonly used by males with EDD and two, rubber-like artificial vagina devices from the sex novelty field. The problem with these antecedent devices is that they do not provide a realistic simultaneous sucking and membrane-like tactile sensation for the male sexual organ as obtained from actual sexual intercourse with a female. The antecedent hard plastic suction pumps in particular do not provide for the pleasurable movement and friction of the male sexual organ within a membrane-like lining. Further, these antecedent devices are expensive, cumbersome, large devices that usually require two hands to operate. These antecedent devices are complex and not easily operated. There is thus a need for a new simple device that provides a realistic simultaneous variable sucking and membrane-like tactile sensation for the male sexual organ. Further, there is a need for a male sexual organ stimulator that also provides for the pleasurable movement and friction sensation to the male sexual organ. Further, there is a need for a device that fits snugly around the male sexual organ for optimum erotic stimulation.

### SUMMARY OF THE INVENTION

The present invention relates to a device that is an erotic stimulator for the male sexual organ and relates to a device that enables males with EDD to obtain an erection to accomplish sexual intercourse. The present invention includes a soft plastic cylindrical container with one open end to receive the male sexual organ. The present invention further includes condoms with tap water sealed inside each which are then inserted into the aforementioned soft plastic cylindrical container to provide a membrane-like variable tactile surface for the erotic stimulation of the male sexual organ. The present invention is also used by males to obtain an erection in the male sexual organ in order to accomplish sexual intercourse.

I claim:

1. A device used to erotically stimulate the male sexual organ and used to obtain an erection to accomplish sexual intercourse by males with Erectile Dysfunction Disorder (EDD) comprising:
a) An approximate six-inch long, 2-inch diameter soft plastic Clairol hair color applicator container or equivalent with an open end which serves as a cavity for receiving the male sexual organ
b) Two standard condoms with approximately one average mouthful of tap water contained and sealed within each said condom
c) said condoms containing tap water inserted into the aforementioned container and serving as a realistic membrane-like, free-floating variable tactile surface for stimulating the male sexual organ
d) lubricant such as hand/body lotion or KY Jelly inserted into the aforementioned hair color applicator container which has the aforementioned condoms inserted within
e) said device being simple to operate with one hand; compact, inexpensive, easily assembled, washable, indefinitely reusable, inconspicuous, and optionally disposable.
